(19) United States
(12) Patent Application Publication
(10) Pub. No.: US 2026/0315125 A1
(43) Pub. Date: Oct. 15, 2026

(54) SYSTEM AND METHOD FOR A MEDICAL IMPLANT WITH INTEGRATED PROPULSORS

(71) Applicant: Randall F. Lee, Southlake, TX (US)

(72) Inventors: Randall F. Lee, Southlake, TX (US); Ian A. Lee, Carrollton, TX (US); Matthew Shomper, Xenia, OH (US); Lisa Ferrara, Hendersonville, NC (US)

(73) Assignee: Randall F. Lee, Southlake, TX (US)

(21) Appl. No.: 19/573,070

(22) Filed: Mar. 20, 2026

Related U.S. Application Data
(63) Continuation of application No. PCT/US2025/028528, filed on May 8, 2025.
(60) Provisional application No. 63/644,222, filed on May 8, 2024.
(60) Provisional application No. 63/677,230, filed on Jul. 30, 2024.

(51) Int. Cl. A61B 17/86; A61F 2/30

(52) U.S. Cl. CPC A61B 17/86; A61F 2/30767; A61B 2017/8655; A61F 2002/30289; A61F 2002/3093

(57) ABSTRACT

Disclosed are embodiments of medical implants containing a chassis and an integrated propulsor system that allow for smooth placement within the bone in addition to compressing the bone both during insertion and afterward.

### CROSS-REFERENCE TO RELATED APPLICATIONS

This application is a continuation of International Application No. PCT/US 2025/028528, filed May 8, 2025, entitled “SYSTEM AND METHOD FOR A MEDICAL IMPLANT WITH AN INTEGRATED PROPULSOR,” which claims the benefit of the filing date of U.S. provisional patent application Ser. No. 63/644,222, filed on May 8, 2024, entitled “SYSTEM AND METHOD FOR A MEDICAL IMPLANT WITH AN INTEGRATED PROPULSOR,” and U.S. provisional patent application Ser. No. 63/677,230, filed on Jul. 30, 2024, entitled “SYSTEM AND METHOD FOR A MEDICAL IMPLANT WITH AN INTEGRATED PROPULSOR, the disclosures of which are incorporated herein by reference for all purposes.

### TECHNICAL FIELD

The invention relates, in general, to fixation and/or fusion of surgically implanted medical devices. In particular, the invention relates to medical implantable devices with two or more integrated propulsors.

### BACKGROUND INFORMATION

Over the years, orthopedic surgeons have developed numerous implants and tools for joining bony structures together. Such fixation or joining is desirable when the bony structure is broken due to trauma or iatrogenic action. In other situations, it may be desirable to join two bony structures together to relieve nerve impingement or other medical conditions.

In many situations, bony structures are joined with medical implants, which must be positioned and secured by applying impact forces. In certain situations, impact forces may cause further damage or trauma. Furthermore, the placement of an implant via impact forces may not be precise.

What is needed, therefore, is a medical implant or implantable device that does not rely solely on impact forces for positioning and placement.

These and other features and advantages will be more clearly understood from the following detailed description taken in conjunction with the accompanying drawings. It is important to note that the drawings are not intended to represent the only aspect of the invention. The features and advantages of the present disclosure will be readily apparent to those skilled in the art. While numerous changes may be made by those skilled in the art, such changes are within the spirit of the invention.

### BRIEF DESCRIPTION OF THE DRAWINGS

FIG. 1A is a distal top perspective view of one embodiment of a medical implant.

FIG. 1B is a proximal top perspective view of the medical implant of FIG. 1A

FIG. 1C is a side perspective view of the medical implant of FIG. 1A.

FIG. 1D is a distal top perspective view of a chassis that may be used in several embodiments of the embodiment shown in FIG. 1A.

FIG. 1E is a proximal top perspective view of a chassis that may be used in several embodiments of the embodiment shown in FIG. 1A.

FIG. 1F is a perspective view of a propulsor that may be used in the embodiment illustrated in FIG. 1A.

FIG. 1G is a side view of the propulsor of FIG. 1F.

FIG. 1H is a perspective view of a propulsor that may be used in the embodiment illustrated in FIG. 1A.

FIG. 2A is a distal top perspective view of an alternative embodiment of a medical implant.

FIG. 2B is a proximal top perspective view of the medical implant of FIG. 2A

FIG. 2C is a side perspective view of the medical implant of FIG. 2A

FIG. 2D is a distal top perspective view of a chassis that may be used in several embodiments of the embodiment shown in FIG. 2A.

FIG. 2E is a perspective view of a propulsor that may be used in the embodiment illustrated in FIG. 2A.

FIG. 3A is a partial side perspective view from the front of a propulsor illustrating certain surface details of particular embodiments.

FIG. 3B is a partial section view through the turbine blades of the embodiment illustrated in FIG. 3A.

FIG. 3C is a partial detailed section view through the turbine blades of the embodiment illustrated in FIG. 3A.

FIG. 3D is a partial detailed view of an alternative turbine blades embodiment.

FIG. 3E is a partial side perspective view from the back of a propulsor illustrating certain surface details of particular embodiments.

FIG. 4A is a longitudinal section view of a propulsor which may be used in several of the embodiments of the present invention.

FIG. 4B is a longitudinal section view of the propulsor of FIG. 4A with the interior removed for clarity.

FIG. 5A is a top view of the embodiment illustrated in FIG. 1A, showing some of the forces on the embodiment as the implant is propelled through a medium.

FIG. 5B is a flowchart illustrating one method of inserting the implant.

### DETAILED DESCRIPTION

For the purposes of promoting an understanding of the principles of the present inventions, reference will now be made to the embodiments or examples illustrated in the drawings, and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

When directions, such as upper, lower, top, bottom, clockwise, counter-clockwise, front, or back, are discussed in this disclosure, such directions are meant to only supply reference directions for the illustrated figures and for orientation of components with respect to each other or to illustrate the figures. The directions should not be read to imply actual directions used in any resulting invention or actual use. Under no circumstances should such directions be read to limit or impart any meaning into the claims.

### A Double Propulsor System

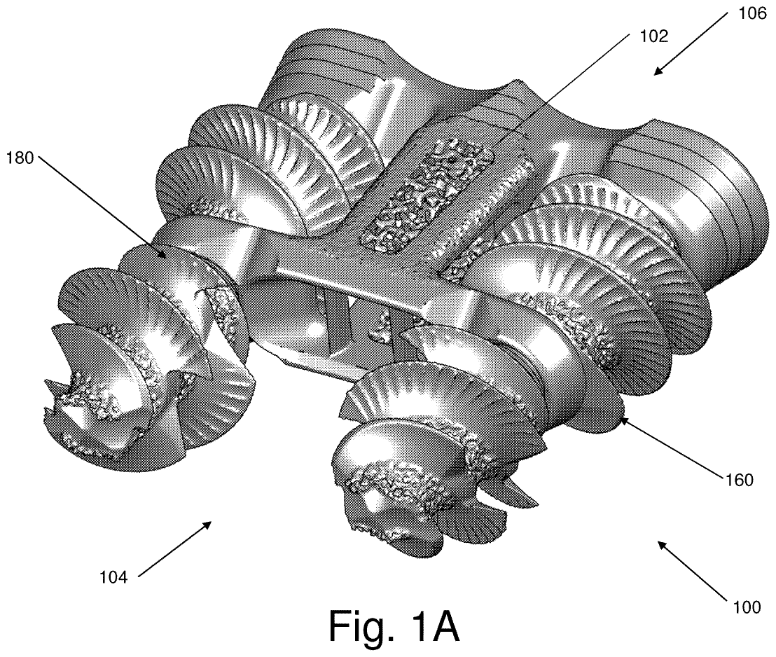

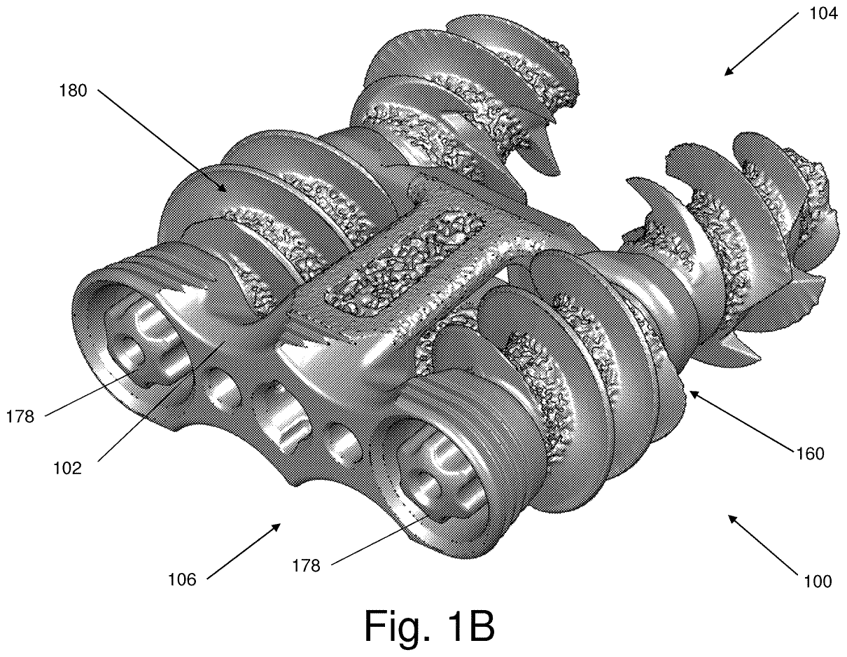

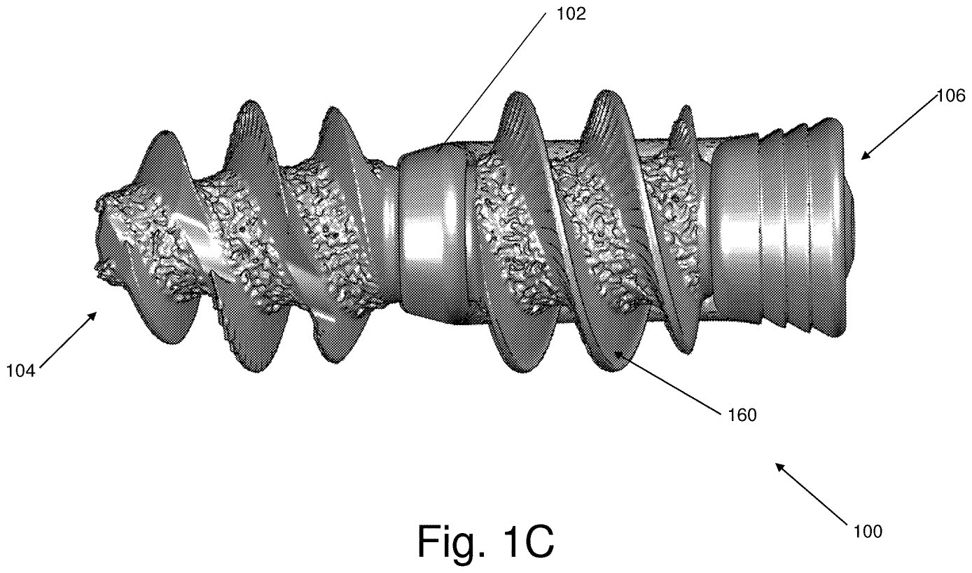

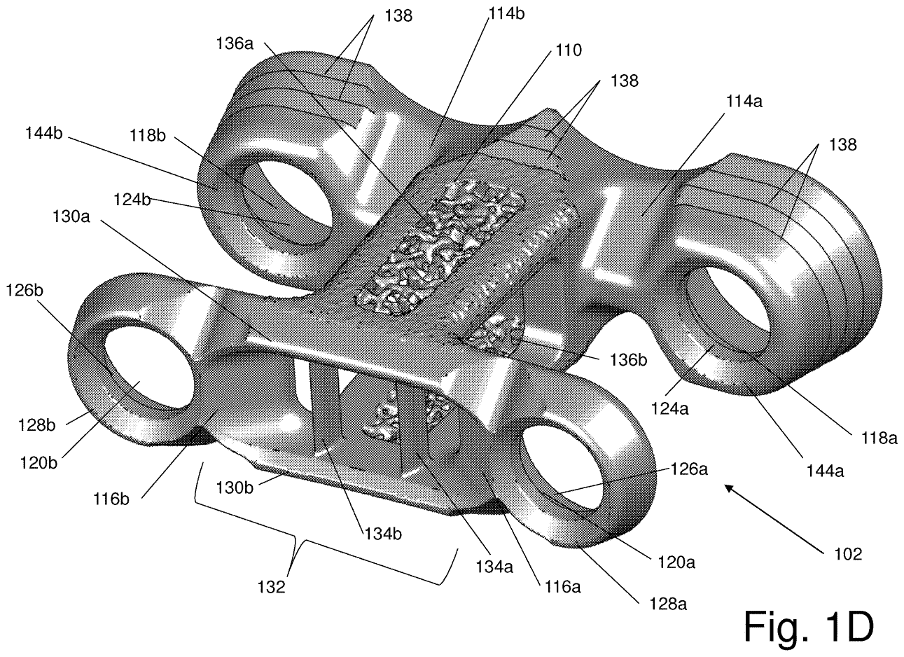

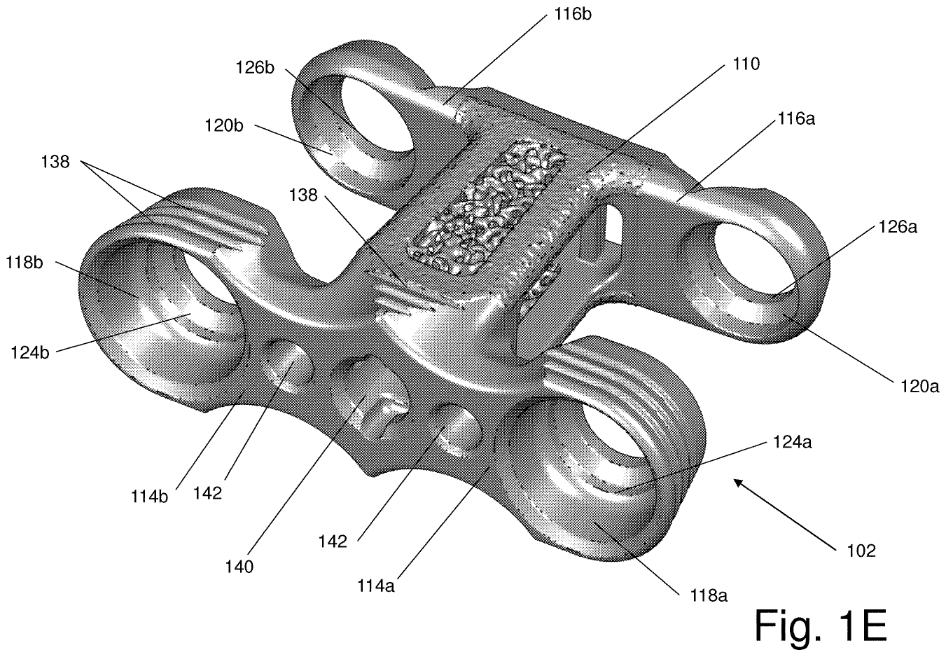

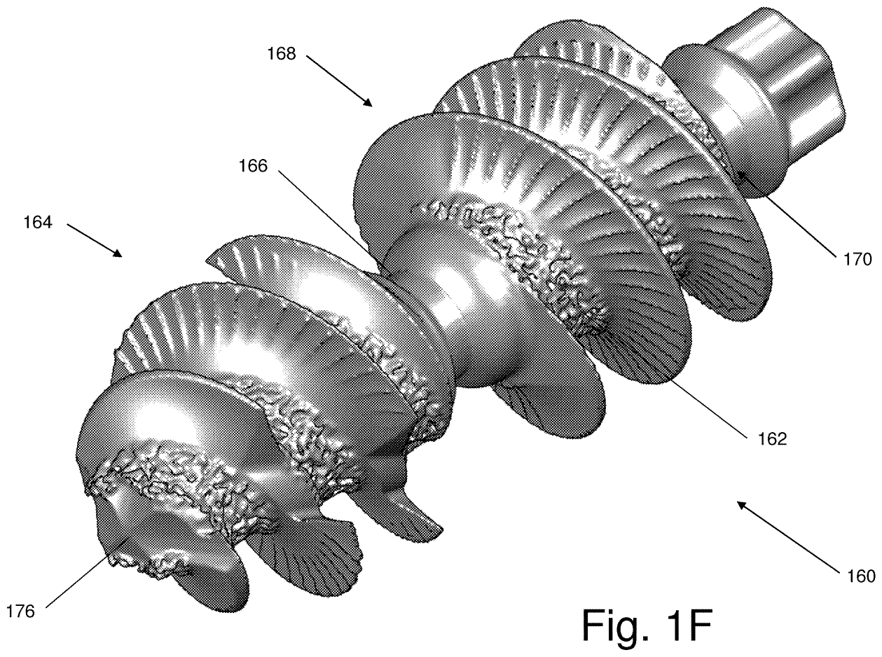

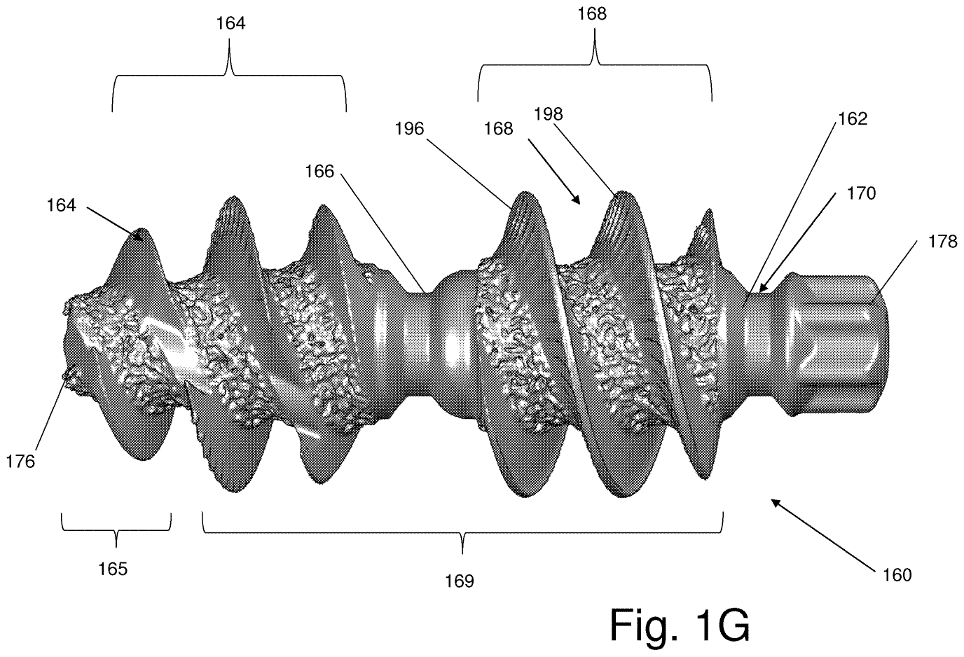

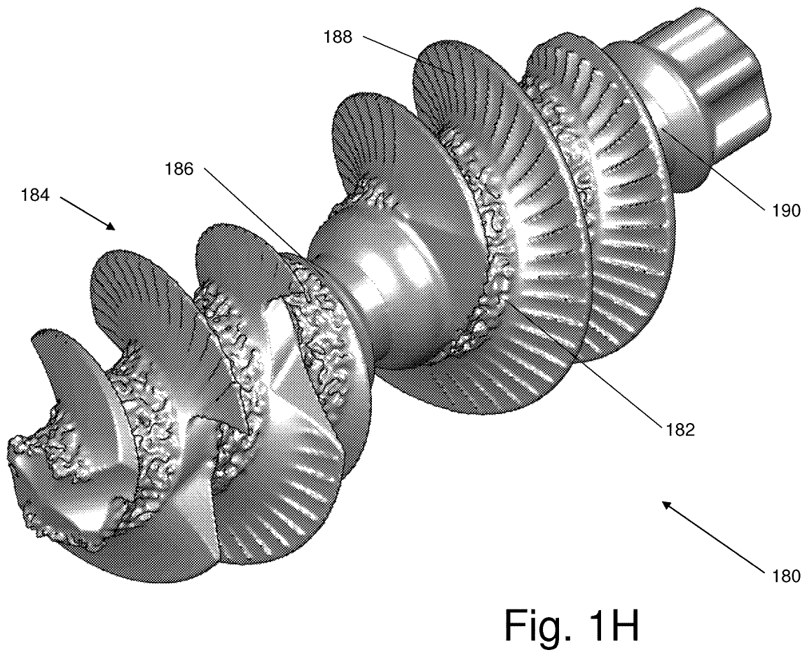

Turning now to FIG. 1A, there is a top perspective view of one embodiment of a medical implant 100 illustrated from a distal perspective. FIG. 1B is a top perspective view of the medical implant 100 illustrated from a proximal perspective. FIG. 1C is a side perspective view of the medical implant 100. As illustrated in FIGS. 1A, 1B, and 1C, the medical implant 100 comprises a chassis 102, a first propulsor 160, and a second propulsor 180.

The implant 100 has a distal end 104 and a proximal end 106. As will be explained below, the chassis 102 provides alignment and structural support for the first propulsor 160 and the second propulsor 180 while allowing the first and second propulsors to rotate about their respective longitudinal axes. In certain embodiments, the first propulsor 160 rotates in an opposite direction from the second propulsor 180. Consequently, the second propulsor 180 has a reverse thread orientation to the first propulsor 160, as illustrated in FIGS. 1A and 1B.

### The Double Propulsor Chassis

FIG. 1D is a top perspective view from the distal end illustrating the example chassis 102 where the first propulsor 160 and the second propulsor 180 have been removed for clarity. Similarly, FIG. 1E is a top perspective view of the chassis 102 from the proximal perspective, with the first propulsor 160 and the second propulsor 180 removed for clarity.

In the illustrative embodiment of FIGS. 1D and 1E, the chassis 102 comprises a center frame or cage 110 having a longitudinal axis or center axis (not shown). In certain embodiments, the cage 110 acts as a retaining chamber for harvested bone material, as explained below. A plurality of proximal extension arms 114a-114b extends out from the center frame 110 in a generally transverse direction to the longitudinal axis. Similarly, a plurality of distal extension arms 116a-116b also extends out from the center frame 110 in a generally transverse direction to the longitudinal axis. In the illustrative embodiment, proximal apertures 118a and 118b are defined within the proximal extension arms 114a and 114b, respectively. Similarly, distal apertures 120a-120b are defined within the distal extension arms 116a and 116b.

In certain embodiments, the apertures 118a and 120a are longitudinally aligned and have a common longitudinal axis (not shown). Similarly, the apertures 118b and 120b are longitudinally aligned and have a common longitudinal axis (not shown). In other embodiments, the longitudinal axes converge at some distal point (not shown) in “front” of the implant 100. In yet other embodiments, the common longitudinal axes of the apertures remain parallel to each other. In yet other embodiments, the longitudinal axes diverge (or converge at some proximal point “behind” the implant 100).

The proximal apertures 118a and 118b define circular bearing surfaces 124a and 124b which are sized to allow the bearing surface 166 of the first propulsor 160 and the bearing surface 186 of the second propulsor 180 to freely rotate when positioned within the respective apertures as illustrated in FIG. 1A. Similarly, the distal apertures 120a and 120b define circular bearing surfaces 126a and 126b which are sized to allow the bearing surface 170 of the first propulsor 160 (See FIG. 1F) and the bearing surface 190 of the second propulsor 180 (See FIG. 1G) to freely rotate when positioned within their respective apertures as illustrated in FIGS. 1A and 1B.

The lateral position of the circular apertures 118a-118b and 120a-120b from the exterior surfaces of the cage 110 allows the respective thread flights 168 and 188 (See FIGS. 1F and 1G) of the propulsors 160 and 180 to rotate freely within the apertures when torque is applied to the propulsors. Additionally, the circular apertures 118a and 118b are close enough to the cage 110 to allow an interaction between the flights 168 and 188 (See FIGS. 1A and 1B) and the surrounding bony tissue (not shown) so that rotating the flights 164, 168 and 184, 188 of the respective propulsors 160 and 180 propels the entire implant 100 forward as described in detail below.

In certain embodiments, distal edges of the chassis 102 and arms 116a-116b are narrowed and/or tapered into cutting surfaces designed to cut and harvest bone as the implant 100 moves into the bony structure (not shown) during the surgical insertion process. Similarly, in some alternative embodiments, the distal or leading edges surrounding the apertures 118a and 118b may be defined by sharp edges 144a and 144b, respectively. In certain embodiments, such sharp edges or features help to cut and wedge the chassis 102 into the intended space without incurring significant force penalties and trauma to the surrounding bone material. For instance, the leading edges of circular distal surfaces forming apertures 120a and 120b are shaped to form cutting edges 128a and 128b as illustrated in FIG. 1A. In certain embodiments, distal blade-like elements 130a-130b of the chassis 102 may function as an “open mouth” area 132 which allows for the harvesting of the bone tissue as the implant 100 moves forward through the bony tissue (not shown). In certain embodiments, vertical or transverse blade like elements 134a and 134b are also designed to cut through bony material in addition to providing structural support for the distal blade-like elements 130a and 130b.

In certain embodiments, walls 136a and 136b of the center frame 110 have fenestrated regions or regions made from porous materials (as illustrated) to allow for bone growth in and around the chassis.

In some illustrated embodiments, teeth like “backward facing” ridges and/or detents 138 may be defined around the perimeter of the chassis 102 and on the exterior surfaces of the chassis 102. These ridges have sharp edges that are angled to allow for forward movement (towards the distal direction), but are designed to catch on bony material to prevent backout during placement once the implant 100 is in place.

In some embodiments, a guide aperture 140 (see FIG. 1E) may be defined on the proximal face of the chassis 102 to allow for placement of a guidewire (not shown) during the surgical insertion process and/or to inject flowable materials into the cannulation, such as biologics, glues, or other osteogenic or osteroretentive material.

In certain embodiments, engagement features and/or apertures 142 (See FIG. 1E) may also be defined on the proximal face of the chassis 102 which are sized and shaped to mate with insertion instruments (not shown) to allow the insertion instrument to rigidly hold the chassis 102 during insertion and placement. In yet other embodiments, such apertures allow for supplemental fixation, such as a screw or nail (not shown) to anchor the implant 100 in place after insertion and positioning.

### The Propulsors

FIG. 1F is a perspective of the first propulsor 160 as viewed from its distal end. FIG. 1G is a side view of the first propulsor 160. The first propulsor 160 includes an elongated body or shaft 162 with a first or distal thread flight 164 extending out from a distal portion of the shaft 162 in a helical manner, a smooth bearing surface 166 of the shaft, a second or proximal thread flight 168 extending out from the shaft 162 in a helical manner, and a second bearing surface 170 of the shaft. As discussed above, the bearing surfaces 166 and 170 are sized to fit with the apertures 118a and 120a (see FIG. 1D), respectively, such that the propulsor 160 can freely rotate within the apertures. In certain embodiments, a proximal end 178 (See FIG. 1B) of the center shaft 162 may be shaped to engage a torque-inducing device (not shown). For instance, in some embodiments, the proximal end 178 may be a torx-shaped head for engaging a similarly shaped driver of an insertion instrument (not shown).

In certain embodiments, a distal end 176 of the center shaft 162 may have an aggressive taper or a threaded region 165 where the shape of the threads or distal surface is designed to drill or cut into the bone or bony tissue. In such embodiments, there may also be a “propeller” region 169, where the shape of the threads or thread form has unique features. The helical threads comprising the propeller region or propeller 169 have unique characteristics and features designed to increase propulsion force to propel the implant fixture 100 in a forward direction while creating compression in the surrounding bony tissue during and after placement. Thus, this helical flight creates greater propulsion and compression. For purposes of this disclosure, this “region of increased propulsion and compression” will be referred to as a propeller region or “propeller” 169.

In certain embodiments, the drilling region 165 may include all of the distal thread flight 164. In such embodiments, then the propeller region 169 is the same as the proximal thread flight 168. In other embodiments, such as the one illustrated in FIG. 1G, the drilling region 165 is only a portion of the first thread flight 164. Consequently, the propeller region encompasses all of the proximal thread flight 168 and a portion of the thread flight 164.

In certain embodiments, the propeller region 169 may extend to the entire length of the propulsor. In other embodiments, there may be two, three, or even four flights (not shown) with varying thread pitches and crest diameters surrounding the center shaft 162, depending on the specific application, length of the implant, or other factors.

In the illustrative embodiment, the threaded region 165 is immediately after the distal end 176. As explained above, in certain embodiments, this threaded region 165 provides the requisite interaction with bony tissue (not shown) to propel the implant 100 forward until the propeller region 169 can interact with the bony tissue-at which time, one or both the propeller 169 and/or the threaded distal region 165 may propel the implant forward. The thread pitch and diameter may change between regions or even within regions, depending on the specific situation thought to be encountered. For instance, some conditions may require a more aggressive cutting flight, while some conditions may require more of a propeller action.

As best seen in FIG. 1G, there is presented an aggressive helical thread form geometry for the propeller 169 that is designed to increase the propulsion force to advance the implant 100 during placement and pull adjacent bony material into the retaining cavity or cage 110 of the chassis 102 (See FIG. 1A). As illustrated in FIG. 1G, the aggressive propeller design is evidenced by relatively high crests of the threads in the propeller region 169 showing a significant difference between the major and the minor diameters—which are relatively large compared to the prior art, such as cortical bone thread. This difference helps to maximize the propulsive force since the propeller 169 must not only drag the implant 100 into the bone but also drag adjacent bone material into the retaining cavity or cage 110 of the chassis 102 to provide the additional compression of the bone material.

In certain embodiments, the pitch and major diameter of the thread form of the propeller 169 (and/or the cutting region 165) may vary along the shaft 162 to further enhance compression as the propulsor 160 is driven into bony material. For instance, a distal thread 196 major diameter of the propeller region 169 may be slightly larger than the major diameter of the proximally positioned thread 198 of the propeller region 169 (See FIG. 1G) to create subtle additional compression as the propulsor 160 is driven into the bony material. Similarly, a major diameter of a distal thread in the cutting region 165 may be slightly larger than a proximal thread major diameter to create subtle additional compression as the propulsor 160 is driven into the bony material.

Turning now to FIG. 1H, which illustrates the second propulsor 180. In the illustrated embodiment, the second propulsor 180 includes an elongated body or shaft 182 with a first or distal flight 184 extending outwards from the shaft 182 in a helical manner, a smooth bearing surface 186 of the shaft, a second or proximal flight 188 (or propeller) extending out from the shaft 182 in a helical manner, and a second bearing surface 190 of the shaft. As discussed above, the bearing surfaces 186 and 190 are sized to fit with the apertures 118b and 120b (see FIG. 1D), respectively, such that the propulsor 180 can freely rotate within the apertures.

The second propulsor 180 is similar to the propulsor 160 except that the helical thread flights of the propulsor 180 are in a reversed helical orientation (e.g., in a counter-clockwise orientation versus a clockwise orientation). Consequently, a detailed discussion of the propulsor 180 will not be presented here for the sake of brevity and clarity.

It should be noted that although two propulsors are described herein, the scope of the present invention contemplates an implant with a chassis and any number of propulsors. For instance, the following is a description of a single propulsor system.

### A Single Propulsor System

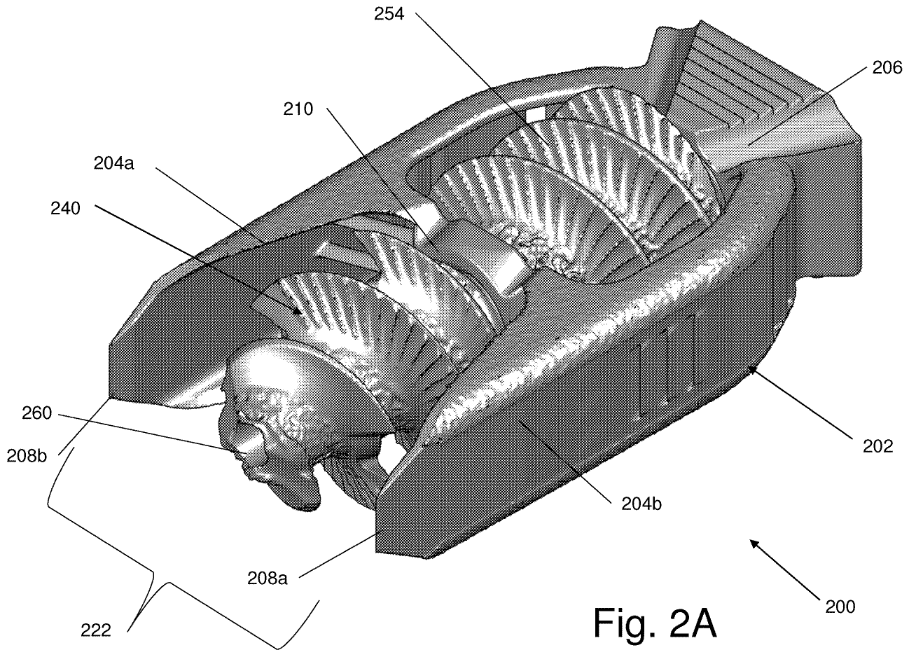

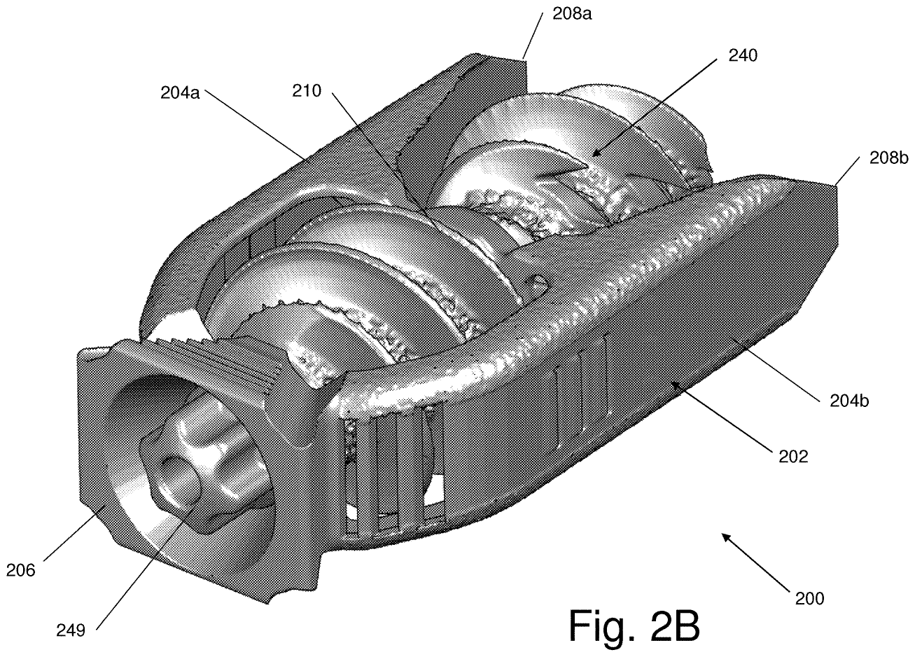

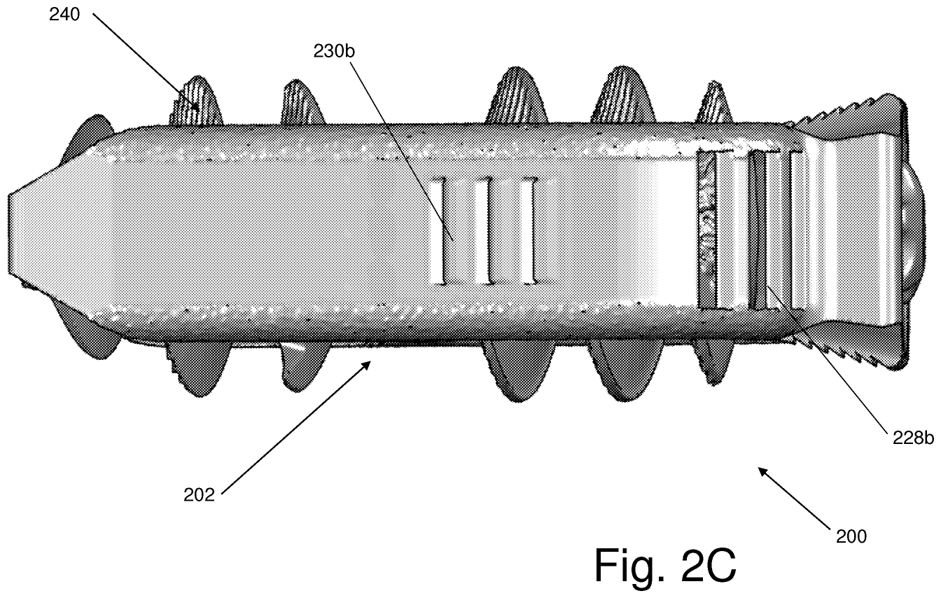

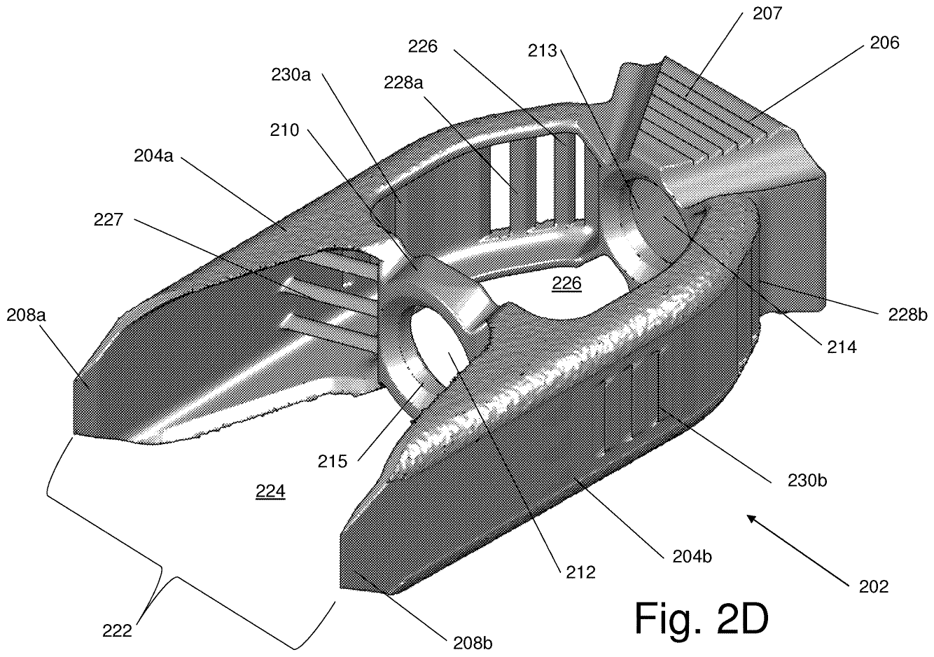

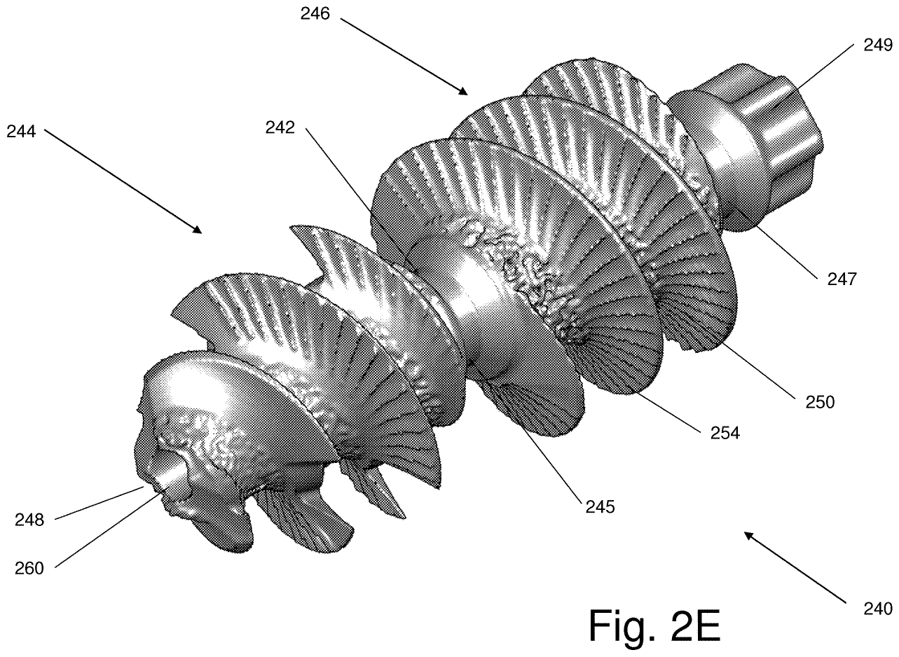

Turning now to FIG. 2A, there is a top distal perspective view of an alternative embodiment of a medical implant 200 comprising a chassis 202 and a single propulsor 240. FIG. 2B is a top proximal perspective view of medical implant 200. In contrast, FIG. 2C is a side perspective view of one embodiment of the medical implant 200. In FIG. 2D, the propulsor 240 has been removed for clarity. Note that the propulsor 240 is similar to the propulsors 160 to 180 discussed above, with certain differences explained below.

As in the previous embodiments, the chassis 202 provides alignment and structural support for the propulsor 240 while allowing the propulsor to rotate about its respective longitudinal or center axes. As will be explained below, when the propulsor 240 rotates in an appropriate direction, the propulsor pulls the chassis 202 in a forward direction.

### The Single Propulsor Chassis

Turning now to FIGS. 2A through 2D, it can be seen that the chassis 202 is generally U-shaped and comprises two arms 204a and 204b extending distally from a proximal end or base 206. Distal tips 208a and 208b of the arms 204a and 204b are narrowed and/or tapered into cutting surfaces to more easily move through a medium, such as a bony structure. In certain embodiments, the chassis 202 may be fenestrated or made from porous materials to allow for bone growth in and around the chassis.

In certain embodiments, a supporting bridge 210 spans between the first arm 204a and the second arm 204b. The supporting bridge 210 helps to support and stabilize the two arms 204a and 204b as the arms engage bony material. The supporting bridge 210 defines a circular aperture 212, which includes a bearing surface 215 (FIG. 2D) for supporting and interacting with a smooth surface bearing surface 245 of the propulsor 240 (See FIG. 2E). A second circular aperture 214 may be defined within the proximal bearing base 206. The second center aperture 214 also includes a bearing surface 213 for supporting and interacting with a smooth proximal surface 247 (See FIG. 2E) defined adjacent to a proximal end 249 of the propulsor 240. The circular apertures 212 and 214 are sized to allow a shaft 242 of the propulsor 240 to rotate about its longitudinal axes with respect to the chassis 202 while the interior surfaces or faces of the apertures provide bearing surfaces for the shaft 242.

The lateral position of circular apertures 212 and 214 from the interior face of the arms 204a and 204b allows threads 254 of the propulsor 240 to clear the arms 204a and 204b so that the propulsor 240 can rotate freely when torque is applied to the propulsor 240. However, the arms 204a and 204b are positioned close enough to still allow for an interaction between the threads 254 and the surrounding bony tissue (not shown) so that rotating the threads of the propulsor 240 propels the entire implant 200 forward as described below.

In certain embodiments, bearing surfaces or detents or other such features (not shown) may be defined in the proximal side of the proximal base 206 to allow an insertion instrument to rigidly hold the chassis 202. In certain embodiments, the proximal aperture (not shown) provides for supplemental fixation, such as a screw or nail (not shown) to anchor the implant 200 in place after positioning.

In some embodiments, most, if not all, forward or distal-facing surfaces (interior and exterior) are tapered into cutting surfaces designed to cut and harvest bone as the implant moves into the bony structure. For instance, the distal edges of the central bearing block or supporting bridge 210 and supporting arms 227 (FIG. 2D) have sharp edges to cut into bony material without incurring significant force penalties and trauma to the surrounding bone material.

In certain embodiments, there may be side vents or lateral gills 230a and on the opposing side lateral grills 230b, which allow for additional harvesting of adjacent bone material to enter the proximal chamber 226. There may also be proximal vents 228a and 228b to allow for some of the bony material to escape from the retaining cavity 226 if the pressure on the compressed bony material becomes too great to allow for forward movement. Furthermore, in certain embodiments, some surfaces have proximal facing sharp edges 207 to prevent backout and to wedge the chassis into the intended space.

### The Propulsor

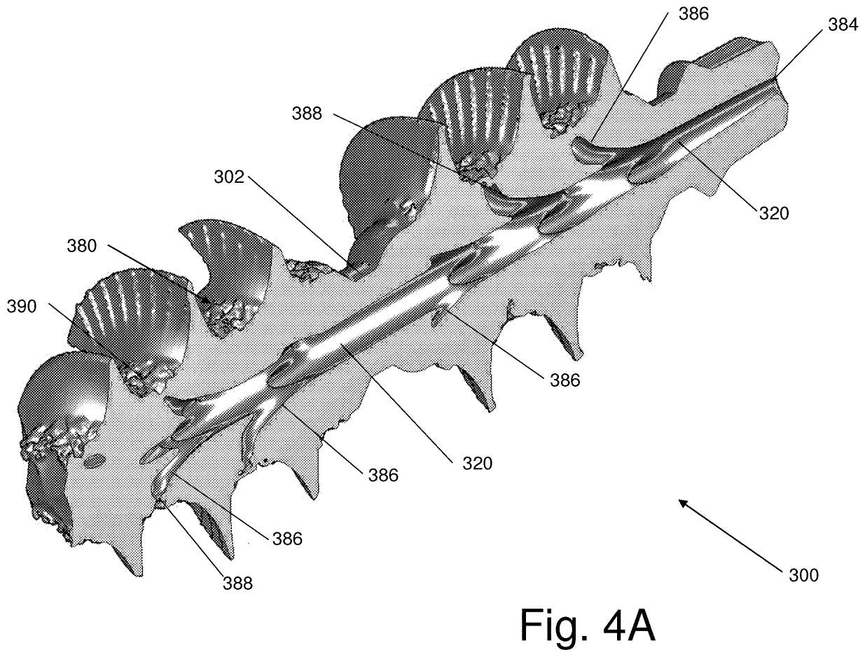

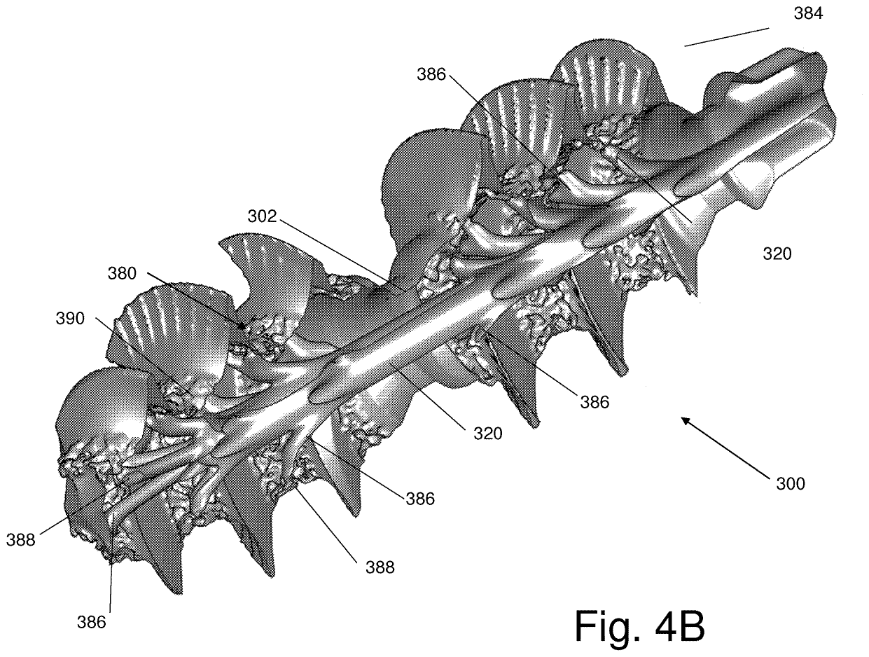

FIG. 2E is a perspective view of one embodiment of the propulsor 240 which may be used with the chassis 202 discussed above. The propulsor 240 is similar to the propulsors discussed above, except that in some embodiments, the longitudinal shaft 242 may be fully cannulated with a central bore 260 to allow for the placement of a guidewire during the implantation process or to inject flowable materials into the cannulation, such as biologics, glues, or other osteogenic or osteroretentive material as described below in reference to FIGS. 4A and 4B.

As with the previous embodiments, the propulsor 240 comprises a center longitudinal shaft 242 that may include two general divisions: a distal portion 244 and a proximal portion 246. In certain embodiments, a smooth bearing surface 245 separates the distal portion 244 from the proximal portion 246.

In certain embodiments, a distal end 248 of the center shaft 242 may have an aggressive taper or cutting surface defined therein to cut through the bony tissue and pull the implant 200 through a medium, such as a bony tissue (not shown). In certain embodiments, a proximal end 249 of the center shaft 242 may be shaped to engage a torque-inducing device (e.g., see FIG. 2B). For instance, in some embodiments, the torque engagement feature may be a 5 mm hex shape for engaging with a 5 mm hex shaped socket driver of an insertion instrument.

As in the previous embodiments, a helical propeller or flight 250 may be coupled to an exterior surface of the proximal portion 246. The propeller 250 is similar to the propeller 169 above. Reference should be made to the discussion regarding FIG. 1G for a complete description regarding the propeller 250.

As in the previous propulsor embodiments, there is illustrated an aggressive screw thread geometry for a thread flight or propeller 250, which is designed to increase the propulsion force to advance the implant 200 during placement and pull adjacent bony material into the retaining cavities 224 and 226 of the chassis 202. The aggressive propeller design is evidenced by relatively high crests of the propulsors, showing a large difference between the major and the minor diameters, as discussed previously. Furthermore, as in the previous embodiments, the pitch of the thread-form of the propeller 250 may vary along the shaft to further enhance compression along the propulsor 240.

As with the previous embodiment, certain embodiments illustrated in FIGS. 2A through 2E include a propeller-like or turbine-like screw design that is intended for posterior implantation into various foot and the sacroiliac joint spaces. Such embodiments are designed to increase propulsion force when moving through bony material. As explained above, in certain embodiments, there are lateral gills or vents to allow bone to be pulled into the device. Certain embodiments also have compression aspects and a superior-inferior method to wedge itself in place. In sum, certain embodiments are dual-acting, accomplishing both insertion and compression with a continuous smooth force.

In certain embodiments, the distal blade-like ends 208a-208b of the arms 204a-104b of the chassis 202 may function as an “open mouth” area 222 (FIG. 2D) which allows for the harvesting of the bone tissue as the implant 200 moves forward through the bony tissue.

This forward movement tends to push the bony tissue in front of the implant 200 through the aperture or mouth 222 and into a first retaining cavity 224 (See FIG. 2D). Once the bony tissue is in the first retaining cavity 224, additional movement and bony material may push the bony material into a second retaining cavity 226 due to relatively large openings (formed by the support arms 227) between the first cavity 224 and the second cavity 226.

Additionally, the rotation of the flight 250 about the shaft 242 also cuts through the bony tissue on the exterior side of the implant 200. Excess bony tissue from the side is rotated by the flight 250 and into the retaining cavity 226. Thus, the tissue entering through the mouth 222 and the tissue entering through the sides by the rotation of the flight 250 “self-fill” the retaining cavity 226 with local tissue graft material and compresses that same harvested material within the retaining cavity as the implant is propelled forward. Such self-grafting within the implant 200 may encourage bridging bony fusion.

As the medical implant 200 advances further along its intended insertion path, additional bony tissue is harvested into the retaining chambers, as explained above. The additional harvesting or filling of the retaining cavities 224 and 226 may cause a compaction of the bony tissue inside the retaining cavity. Furthermore, the protruding side vents 230a-230b macerate the surrounding bone and self-harvest the surrounding bone material (i.e., pushes the bone through the side vents into the proximal chamber)

### Thread Form Surface Features

In addition to the unique features of the implants discussed above, each of the propulsors 160, 180, and 240 may incorporate several unique surface and internal features. For simplicity, these features will be discussed in terms of an example propulsor 300. However, any of these features may be incorporated into the propulsors discussed above and still be within the scope of this invention. Additionally, the surface features discussed below may be incorporated into aggressive cutting regions, such as region 165 (discussed above) or the propeller region 169.

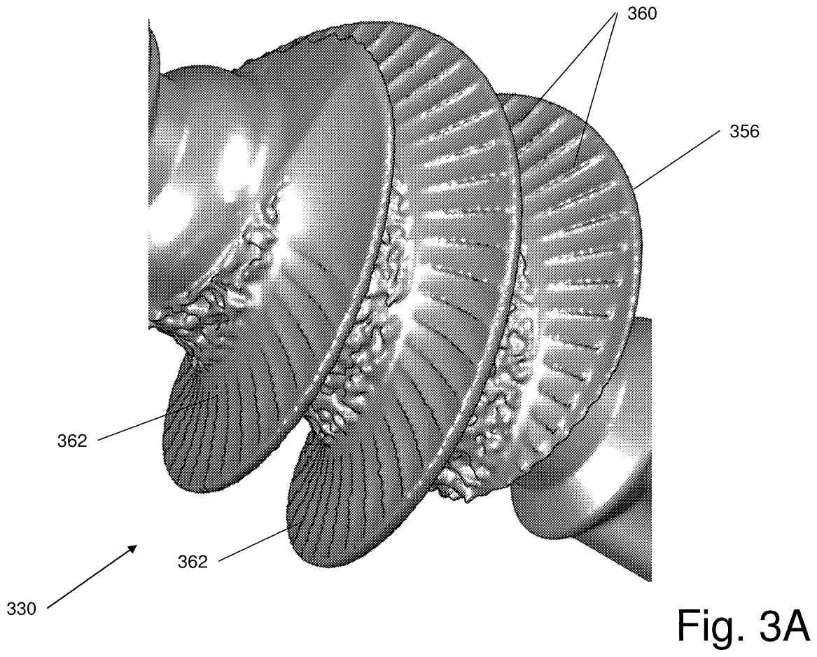

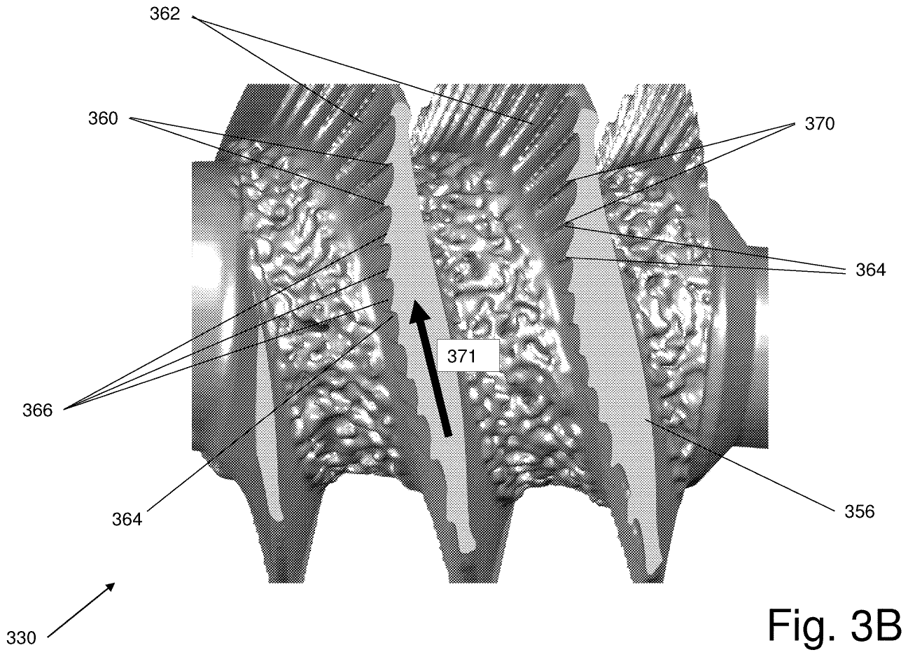

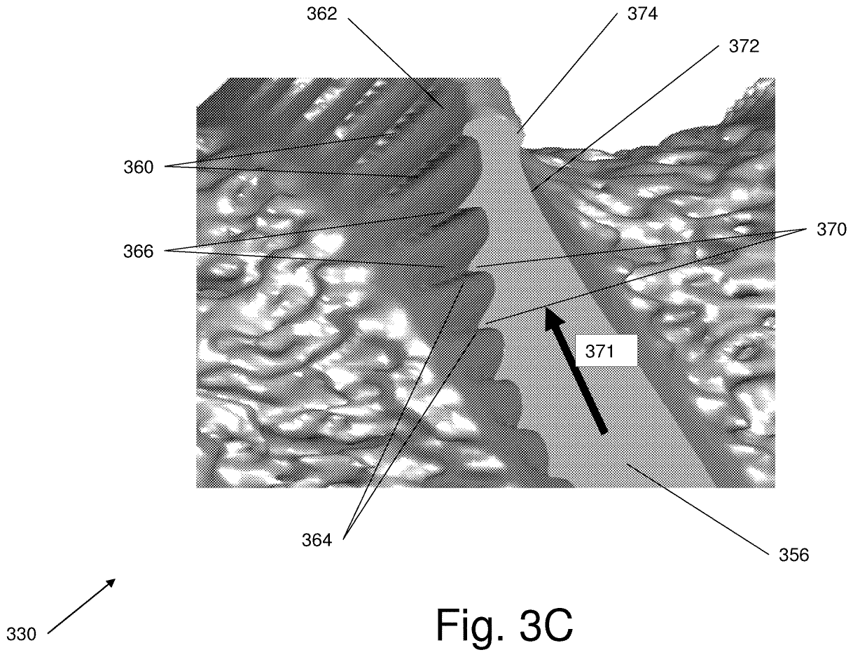

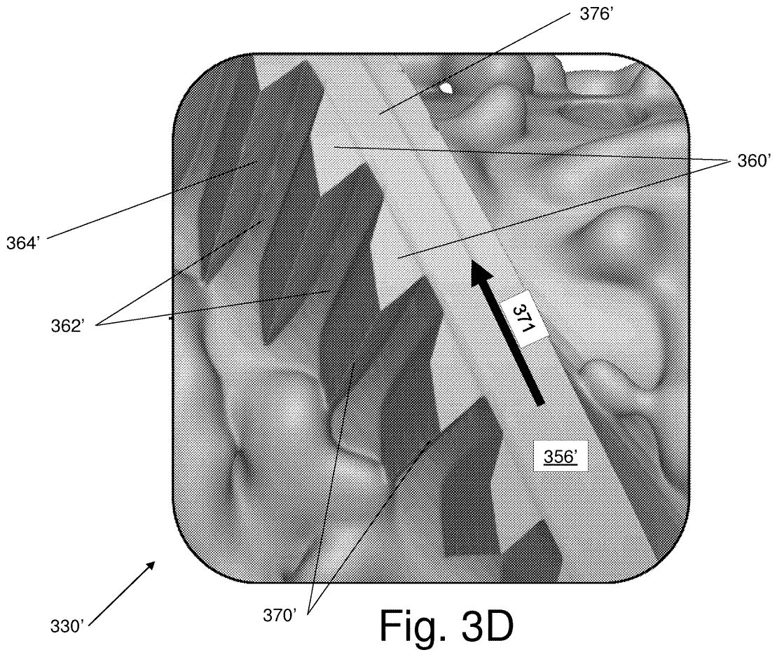

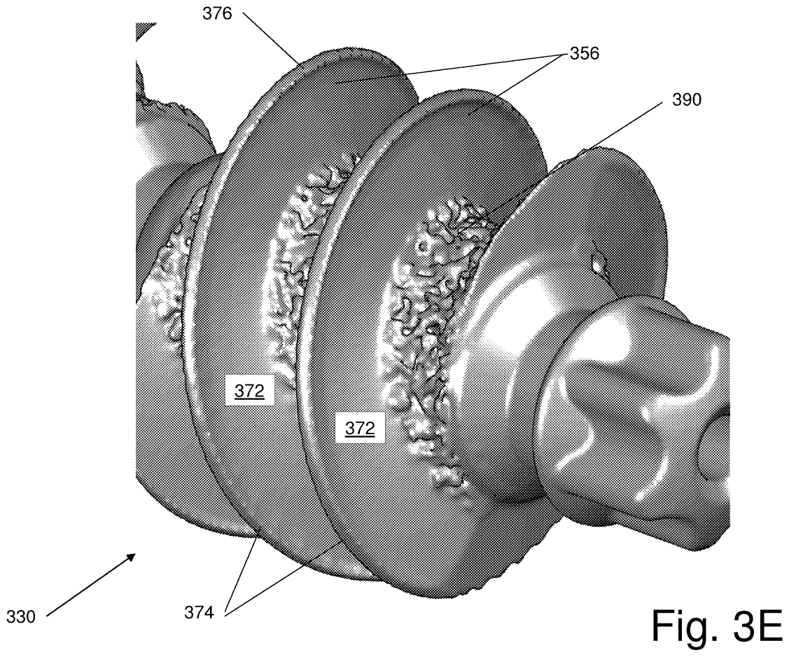

FIG. 3A is a partial side perspective view of a surface detail of one of the examples of a propulsor 300 and specifically a propeller or propeller region 330 (similar to the propeller region 169 discussed above) illustrating certain distal facing or front side surface details. In contrast, FIG. 3B is a partial section view showing a cut through the threads of the propeller region 330. FIG. 3C is a detailed sectional view of the thread forms where the longitudinal direction has been expanded for illustrative purposes to show additional details.

As illustrated in FIGS. 3A through 3C, in certain embodiments, there may be “pawls” or radial blades 360 projecting from the front or distal surfaces 362 of the threads 356 of the propeller 330 to create a turbine-like effect. As illustrated, in certain embodiments, the pawls 360 extend outwardly from the direction of the shaft in a somewhat radial manner, generally transverse to the circumferential direction of the thread of the propeller 330. In section, as can be seen in FIGS. 3B and 3C, the pawls 360 are generally triangular in cross-sectional shape, having a leading side or surface 366 pointed away from the direction of travel (arrow 371) and a back side or surface 364 meeting the leading side 366 to form a sharp edge 370. In certain embodiments, the sharp edge 370 may be at a predetermined angle (e.g., 20 degrees) relative to the distal surface 362 of the thread 356 and projects in a generally opposite direction from the forward rotation direction 371 (during placement of the propulsor).

The geometry of the pawls 360 enables rotation in a single rotational direction (forward rotational travel 371) and prevents backout rotation, similar to a ratchet and pawl mechanism, because the sharp edges 370 will catch on the surrounding bony structure if the direction of travel is reversed. In certain embodiments, there may be subtle pitch changes in the pawls 360 along the threads, as best illustrated in FIG. 3C, which will result in increased compression of the bony material.

Focusing now on FIG. 3C, there is a detailed section view cut through one of the threads 356. In certain embodiments, a backside surface 364 of the pawl 360 is curved. With the curved back surface, as the thread 356 is rotated, additional pressure is applied against the bony material, creating a turbine-like effect. In other words, as the propulsor 300 rotates into position, the pawls 360 may exert constant pressure even at low rotational speeds. The pressure may increase slightly as the propulsor 300 progresses so that the propulsor 300 creates compression and retains itself as it is placed. With the curved back surface, as the thread 356 is rotated, additional pressure is applied against the bony material, creating a turbine-like effect. In other words, as the propulsor 300 is rotating into position, the pawls 360 may exert constant pressure even at low rotation speeds. The pressure may increase slightly as the propulsor 300 progresses so that the propulsor 300 creates compression and retains itself as it is placed. Furthermore, as rotation occurs, subtle pitch changes will occur along the threads which results in an increase in compression and the “scooped” segments created by the backside of the pawls maximizes surface area for bone ingrowth after placement.

FIG. 3D illustrates an alternative embodiment for the pawl and turbine system on an alternative thread and shows a perimeter edge 376′ of a single thread 356′ of an alternative propeller region 330′. As illustrated in FIG. 3D, the pawls or radial blades 360′ may be more defined than in the previous embodiment discussed above. Similar to pawls 360 discussed above, the pawls 360′ project from the front or distal surfaces 362′ of the thread 356′ to create a turbine-like effect. As illustrated, in certain embodiments, the pawls 360′ extend from the surface 362′ of the thread 356′ towards the distal end of the shaft 302 and generally transverse to the circumferential direction of the thread of the propeller 330. In section, as can be seen in FIG. 3D, the pawls 360′ form a sharp edge 370', which faces away from the rotational direction of travel indicated by arrow 371. In certain embodiments, the sharp edge 370′ may be at a predetermined angle (e.g. 30 degrees) relative to the distal surface 362′ of the thread 356′.

The geometry of the pawls 360′ enables rotation in a single rotational direction 371 (forward rotational travel) and prevents backout, similar to the system described above because the sharp edges 370′ will catch on the surrounding bony structure if the direction is reversed. In certain embodiments, there may be subtle pitch changes along the threads, which will result in increased compression of the bony material. In certain embodiments, the pawls may be evenly spaced throughout the faces of the threads. In other embodiments, the pawls may be spaced differently on the distal and proximal thread portions. For instance, the pawl spacing may increase along the thread flight from the distal threads to the proximal threads. In another embodiment, the pawl spacing may decrease along the thread flight from the distal to proximal threads. In addition, the pawl depth (from the face of the thread) and radial angle may be adjusted as necessary for the specific application. In yet other embodiments, the cross-section shape of the pawls may be a right triangle, an isosceles triangle, or another polygon. Furthermore, in some embodiments, the pawl projection path in the radial direction may be straight, as illustrated, or curved.

In certain embodiments, in cross-section view normal to the longitudinal direction, a backside surface 364′ of the pawl 360′ is curved or straight. The back side surface 364′ allows additional bone harvesting because of a cupping effect, which may also create subtle compression in the harvested bony material, while the thread 356′ of the propeller 330 is rotating to position the propulsor 300. Furthermore, as the thread 356′ is rotated, additional pressure is applied against the bony material, creating a turbine-like effect as well as compression.

FIG. 3E is a partial side perspective view of a propeller 330 illustrating certain proximal facing or backside surface details of the threads 356. Turning now to FIG. 3E and FIG. 3C, the back side or proximal face 372 of the thread 356 is illustrated with a circumferential rim 374 projecting in a proximal direction around the perimeter 376 of the thread 356. Furthermore, in some embodiments, the proximal face 372 may be curved in a concave manner, creating a cupping effect. During rotation occurring during implant placement, the rim 374 and the slight cupping may gather additional bony material between the threads, causing a pressure wave within the bony material, resulting in additional compression of the bony material. This “backside” geometry wedges the propulsor into the bony material, resulting in increased compression of the bony material.

### Flowable Material Distribution System

FIG. 4A is an isometric section view of the propulsor 300. In contrast, FIG. 4B is an isometric section view of the propulsor 300 where the interior solid material is shown in a transparent manner to illustrate certain interior channels, cannulas, structures and details defined within a shaft 302 of the propulsor.

A main central bore or cannula 320 runs along the propulsor's longitudinal or center axis. In propulsors such as the propulsor 240 discussed above, the main cannula 320 may be used with guide wires for accurate placement of the propulsor under fluoroscopy, as is known in the art. However, in propulsors 160 and 180 discussed above, the main cannula 320 may be part of an internal labyrinth structure 380 having a branched, tree-like appearance for distributing biologics and bone growth after implant placement. In certain embodiments, the structure or labyrinth 380 is formed from a plurality of tubules or channels 386 branching off the central cannula 320. The tubules 386 may be internally printed and flow from the center cannula 320 to apertures 388 defined on the surface of the shaft 302 and, in some embodiments, to the threads of the propulsor 300.

In certain embodiments, the initial diameter or size of the tubules 386 varies along the longitudinal axis to allow for relatively even distribution of the flowable biologic material. For instance, distal tubules 386 may have larger diameters where the pressure in the center cannula 320 is less to allow the biologics to flow evenly throughout the entire structure. In certain embodiments, the tubules branch or split into smaller tubules and exit at the minor diameter of the propulsor. In yet other embodiments, the tubules 386 extend into the thread forms (not shown). The tubule size and distribution may be customized for optimal graft flow—depending on the application and the flowability of the injected material.

In certain embodiments, pressurized biological material, such as treated cadaverous bone material, may be injected into the center cannula 320 via an opening 384 formed in the proximal end 249 from a syringe or another pressure-inducing device. The material will then flow down the center cannula and into the various branches of the labyrinth and, in the illustrative embodiment, out into the shaft. In certain situations, certain medicines, such as analgesics or antibiotics, may also be injected into the center cannula to relieve post-operative discomfort and/or to prevent infection.

In certain embodiments, as best illustrated in FIG. 3E, there is a porous lattice or fenestrated structure 390 (which may be organic or irregular in shape and size) that may be 3D printed around the shaft 302 or minor diameter of the propulsor 300 to encourage bone growth. Such a lattice structure may be organic and irregular in shape, as illustrated in FIG. 3E. In embodiments that include the internal labyrinth, the channels 386 will end in this lattice 390 (or slightly beyond the lattice) to further encourage bone growth around the shaft.

### Manufacturing Methods and Materials

In certain embodiments, the implants (such as implant 100) may be manufactured utilizing 3D printing, where the implant is printed as a relatively complete assembly incorporating the propulsors 160 and 180 into the respective chassis. Such embodiments may then be finalized with standard machining methods to clean up or add various surfaces and features. In yet other embodiments, the chassis or propulsors may be separated into multiple pieces so that they may be assembled to form a complete implant. The assembled component pieces may then be joined by manufacturing methods, such as but not limited to pinning, gluing, welding, crimping, or snap-fit.

In certain embodiments, the implants and propulsors discussed above may be fabricated from any number of biocompatible implantable materials, including but not limited to Titanium Alloys (Ti 6Al4V ELI, for example), commercially pure titanium, Chromium Cobalt (Cr—Co) and/or stainless steels. In yet other embodiments, the implants and propulsors may also be manufactured from polymer, including Carbon Fiber Reinforced Polymer (“CFRP”) with a high carbon mass percentage. In some embodiments, the implants (or portions of the implants) may be coated with a bone conducting surface treatment to increase the potential of bone on-, through-, or in-growth.

In certain embodiments, aspects of the invention may include a surgical kit comprising multiple implants of different size ranges.

The compressive features of the thread form discussed above may cause subtle compression during the application of a smooth force. In certain situations, subtle compression may be preferred because the bony structure may not be able to withstand aggressive compression.

### Operation and Method of Use of Certain Embodiments

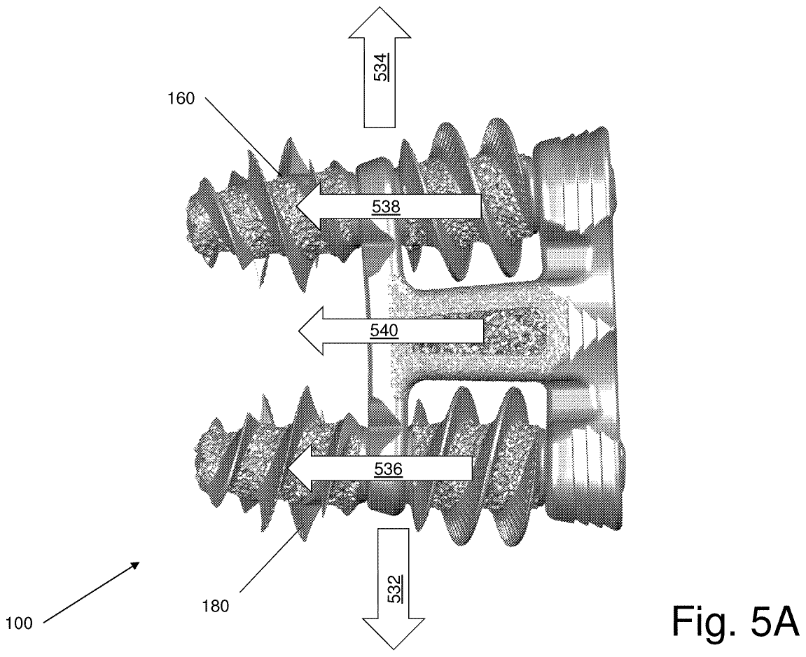

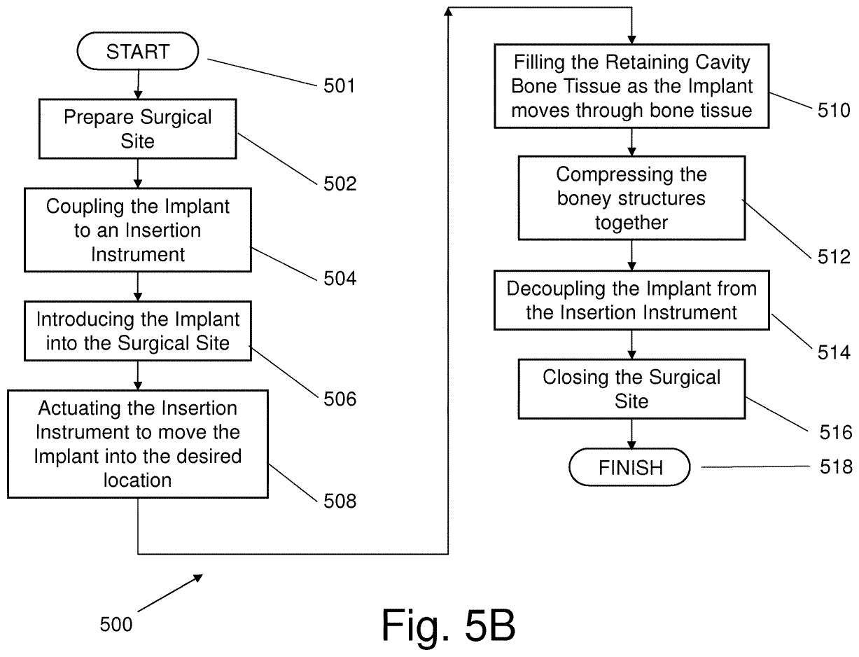

FIG. 5A is a top view of the implant 100 positioned in a medium (not shown), such as bony tissue at a moment when a clockwise rotational force is being applied to the first propulsor 160 while a counter-clockwise rotational force is being applied to the second propulsor 180. The respective rotations cause lateral forces (indicated by arrows 532, 534) and longitudinal forces (indicated by arrows 536, 538) to be applied to the medium, respectively. The lateral forces 532 and 534 are equal in magnitude and opposite in direction. So, they effectively cancel each other. The longitudinal forces 536 and 538, in contrast, are additive in nature and will cause the implant 100 to propel forward within the medium in the direction indicated by the arrow 540.

Referring now to FIG. 5B, the manner of using one embodiment of the dual propulsor embodiment will now be described. FIG. 5B is a flowchart illustrating a surgical method 500 for inserting and positioning certain embodiments of the present invention. The method starts in step 501 and flows to step 502 where a surgical site is selected and prepared for insertion. In certain embodiments, a surgical site may be a facture between two bony structures. In step 504, an implant, such as implant 100 (described above) may be coupled to a torque inducing insertion instrument (not shown). In certain embodiments, the coupling may be made during the manufacturing process if the insertion instrument is designed to be a single use instrument packed in a sterile container. If the insertion instrument is designed to be a multi-use instrument, then the implant 100 may be coupled to the insertion instrument prior to insertion and after the selection of the desired size of the implant.

Once the medical implant 100 is coupled to the insertion instrument, in step 506, the insertion instrument can then be aligned and introduced into the bore structures of the surgical site (in embodiments where pre-drilled bores are necessary or required). In certain embodiments, surgical guidewires may be used to assist in guiding the implant to the desired location. Once aligned, the user may actuate the propulsors 160 and 180 (FIG. 5A) within the implant 100 (step 508) by inducing a torque at the end of the proximal end of the implant, causing a rotation of the propulsors. This rotation will propel the medical implant 100 into the bony structures until the medical implant reaches the desired location. If, for some reason, the medical implant 100 needs to be repositioned during the surgical procedure, the user can induce a torque in the opposite direction-which will cause the implant to reverse direction within the bony tissue so that exact positioning can occur.

As discussed above, the lateral position of the circular apertures 118a-118b, 120a-120b with respect to the cage 110 allows the distal and proximal flights of the propulsors to clear the cage 110 so that the flights can rotate freely when torque is applied to the propulsors 160 and 180. However, the propulsors 160 and 180 are positioned close enough to still allow an interaction between the flights and the surrounding bony tissue (not shown) so that rotating the respective flight of the propulsor propels the entire implant 100 forward.

This forward movement tends to push the bony tissue in front of the implant 100 through the aperture or mouth 132 and into the cage or retaining cavity 110. Additionally, the rotation of the proximal flights 168 and 188 about their respective shafts 162 and 182 also cuts through the bony tissue on the exterior sides of the implant 100. Excess bony tissue from the sides is rotated by the flights 168 and 188 and into the cage 110 which functions as a retaining cavity. Thus, the tissue entering through the mouth 132 and the tissue entering through the sides by the rotation of the flights 168 and 188 to “self-fill” the cage 110 (or retaining cavity) with local tissue graft material and compresses that same harvested material within the retaining cavity as the implant is propelled forward (step 510). Such self-grafting within the implant 100 may encourage bridging bony fusion.

As the medical implant 100 advances further along its intended insertion path, additional bony tissue is harvested into the retaining chamber as explained above. The additional harvesting or filling of the cage 110 may cause a compaction of the harvested tissue inside of the retaining cavity. Additionally, some embodiments with the surface features described above will also cause compression. For instance, subtle pitch changes in the thread flights and backside thread curved or wave geometry will also cause compression.

In certain embodiments with passive or active compression features, the forward movement of the implant 100 through the bony tissue may also cause compression between the bony elements (step 512). In situations where there is a gap between the bony structures, such compression features may close the gap between the bony structures.

Once the medical implant 100 is in the desired location, in step 514, the medical implant 100 may be decoupled from the insertion instrument. In certain embodiments the decoupling may entail pulling on the insertion instrument with enough force to overcome the retaining force on the implant 100 provided by the retaining fingers (not shown). In step 516, the surgical site can then be closed in a traditional manner and the process finishes in step 518.

### Advantages of Certain Embodiments

Certain embodiments also have compression aspects and a superior-inferior method to wedge itself in place. In sum, certain embodiments are dual acting accomplishing both insertion and compression with a continuous smooth force.

Certain embodiments described above include a propeller-like or turbine-like screw design that is intended for situations like posterior implantation into the sacroiliac joint space. Such embodiments are designed to increase propulsion force when moving through bony material. As will be explained below, in certain embodiments, there are lateral gills or vents to allow bone to be pulled into the device.

During implant insertion, the action of the propulsor's rotation compresses the bony elements together. This compression, in turn, produces a material bony element alignment. Furthermore, in certain embodiments, the rotation of the propulsors forces the implant to actively harvest graft into the implant's graft chamber. Continued rotation of the flight of each propulsor also compresses the graft material within this chamber. Furthermore, in certain embodiments, the angular momentum of the flight channels compressed material between and within the flight element itself. Thus, the disclosed implants can be used to compress and stabilize the joint, reducing inflammation and improving function, often leading to pain relief and greater mobility.

The embodiments described above are designed to compress two bones together using a smooth and continuous force, offering numerous advantages during placement and in the post-operative phase. Such a design may enhance stability by securely aligning the bones, reducing micro-movements that might impede healing. This gradual, evenly distributed compression may also stimulate the biological processes essential for healing, as it may facilitate better contact between bone surfaces, which is crucial for osseointegration.

Furthermore, smooth force distribution during placement minimizes stress concentrations at the implantation site, potentially decreasing the risk of bone damage or implant failure due to excessive localized pressure. By providing increased compression over traditional methods, the design may improve the joint stability, promoting faster healing and reducing the time required for bone union.

Moreover, the versatility of such implants means they can be adapted to a wide range of applications including fracture fixation, spinal surgery, joint reconstruction, osteotomies, non-union fractures, and trauma cases.

For instance, the above embodiments may be used when fusing the sacroiliac (SI) joint. The surgeon makes a small incision over the SI joint area. Using fluoroscopic guidance (real-time X-ray imaging), the surgeon carefully navigates instruments to reach the joint. The joint surfaces are then prepared, often by removing any inflamed cartilage and smoothing the surfaces to promote fusion. The various embodiments described above may be implanted and positioned, which will cause immediate compression during placement. Bone graft material may then be placed within the joint space to facilitate bone growth across the joint, as described above. This compression may help stabilize the bones, promote faster and more reliable fusion, and reduce micro-movement that could hinder healing. The above embodiments apply compression and hold the joint in a fixed position, allowing the bone graft to fuse the sacrum and ilium over time. The incision is then closed with sutures.

In fracture fixation, such implants are especially useful for stabilizing long bones like the femur, tibia, and humerus, where secure alignment and effective load transfer are essential for proper healing. In spinal surgery, these implants can be used to stabilize and facilitate fusion of vertebrae, which is often necessary in conditions like degenerative disc disease, scoliosis, or spinal instability, thereby promoting proper alignment and pain relief. For joint reconstruction, such as in knee or hip surgeries, the implants provide necessary stabilization to support post-operative healing and restore joint function.

Moreover, in osteotomies—procedures where bones are intentionally cut and realigned to correct deformities—these implants help maintain the new bone position with continuous compression, ensuring proper healing. They are also valuable in cases of non-union fractures, where bones have failed to heal naturally; the enhanced compression encourages new bone growth and union. During trauma surgeries involving complex or severe fractures, these implants offer reliable fixation and stability to promote timely recovery.

In foot and ankle situations, such implants may be employed during procedures to fuse joints or stabilize fractures, which may provide a strong, uniform compression across the affected bones, which helps in correcting deformities and accelerating healing.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

Any advantages and benefits described may not apply to all embodiments of the invention. When the word “means” is recited in a claim element, Applicant intends for the claim element to fall under 35 USC 112(f). Often a label of one or more words precedes the word “means”. The word or words preceding the word “means” is a label intended to ease referencing of claims elements and is not intended to convey a structural limitation. Such means-plus-function claims are intended to cover not only the structures described herein for performing the function and their structural equivalents, but also equivalent structures. For example, although a nail and a screw have different structures, they are equivalent structures since they both perform the function of fastening. Claims that do not use the word “means” are not intended to fall under 35 USC 112(f).

The foregoing description of the embodiments of the invention has been presented for the purposes of illustration and description. It is not intended to be exhaustive or to limit the invention to the precise form disclosed. Many combinations, modifications and variations are possible in light of the above teaching. For instance, in certain embodiments, each of the above described components and features may be individually or sequentially combined with other components or features and still be within the scope of the present invention. Undescribed embodiments which have interchanged components are still within the scope of the present invention. It is intended that the scope of the invention be limited not by this detailed description, but rather by the claims.

For instance, in some embodiments, there may be a surgical implant comprising a chassis for aligning and holding a first propulsor and a second propulsor, wherein: the first propulsor comprises: a first longitudinal shaft having a first rotational axis; a clockwise-orientated propeller positioned about a first portion of the first longitudinal shaft, and the second propulsor comprising a second longitudinal shaft having a second rotational axis; a counter-clockwise orientated propeller positioned about a portion of the second longitudinal shaft, wherein the clockwise propeller and the counter-clockwise propeller each have a first section of the helical threads and a second section of the helical section of threads wherein the first section of helical threads is positioned distally from the second section of helical threads and the first section of helical threads has a larger major diameter than the second section of helical threads; a circumferential rim projecting from the plurality of the proximal surfaces of the helical threads and wherein the proximal surfaces of the helical threads are curved in a concave manner, and a porous lattice structure formed within the plurality of helical root valleys.

For instance, in some embodiments, there may be a surgical implant comprising a chassis for aligning and holding a first propulsor and a second propulsor, wherein: the first propulsor comprises: a first longitudinal shaft having a first rotational axis; a clockwise-orientated propeller positioned about a first portion of the first longitudinal shaft, and the second propulsor comprising a second longitudinal shaft having a second rotational axis; a counter-clockwise orientated propeller positioned about a portion of the second longitudinal shaft, wherein the clockwise propeller and the counter-clockwise propeller each have: helical threads positioned about a longitudinal portion of the respective shaft forming a plurality of helical root valleys between a plurality of crests of the helical threads, and the helical threads having a plurality of distal surfaces and a plurality of proximal surfaces; a plurality of generally radial pawls projecting from the distal surfaces to create a turbine-like thread-form shape, wherein the first plurality of pawls are angled with respect to the plurality of distal surfaces to allow for forward rotation of the plurality of threads and to resist backward rotation of the plurality of threads; a first section of the helical threads and a second section of the helical section of threads wherein the first section of helical threads is positioned distally from the second section of helical threads and the first section of helical threads has a larger major diameter than the second section of helical threads; a circumferential rim projecting from the plurality of the proximal surfaces of the helical threads and wherein the proximal surfaces of the helical threads are curved in a concave manner, and a porous lattice structure formed within the plurality of helical root valleys.

In other embodiments, there may be a surgical implant comprising a chassis for aligning and holding a first propulsor and a second propulsor, wherein: the first propulsor comprises: a first longitudinal shaft having a first rotational axis; a clockwise-orientated propeller positioned about a first portion of the first longitudinal shaft, and the second propulsor comprising a second longitudinal shaft having a second rotational axis; a counter-clockwise orientated propeller positioned about a portion of the second longitudinal shaft, wherein the clockwise propeller and the counter-clockwise propeller.

In yet other embodiments, there may be a surgical implant comprising a chassis for aligning and holding a single propulsor.

In other embodiments, some of the above propulsors have helical threads positioned about a longitudinal portion of the respective shaft forming a plurality of helical root valleys between a plurality of crests of the helical threads, and the helical threads having a plurality of distal surfaces and a plurality of proximal surfaces and a plurality of generally radial pawls projecting from the distal surfaces to create a turbine-like thread-form shape, wherein the first plurality of pawls are angled with respect to the plurality of distal surfaces to allow for forward rotation of the plurality of threads and to resist backward rotation of the plurality of threads.

In other embodiments, some of the above propulsors also have a first section of the helical threads and a second section of the helical section of threads wherein the first section of helical threads is positioned distally from the second section of helical threads and the first section of helical threads has a larger major diameter than the second section of helical threads.

In other embodiments, some of the above propulsors also have a circumferential rim projecting from the plurality of the proximal surfaces of the helical threads and wherein the proximal surfaces of the helical threads are curved in a concave manner.

In other embodiments, some of the above propulsors also have a porous lattice structure formed within the plurality of helical root valleys.

In other embodiments, a surgical implant comprises a chassis that is configured to retain an anchor, the chassis having first and second arms that are coupled to, and extend from, a base section at a proximal end of the surgical implant, wherein the base section includes a first aperture therein; and a bridge section positioned distally from the base, wherein the bridge section couples the first and second arms and includes a second aperture therein; and the anchor includes a shaft having a proximal end and a distal end with a substantially smooth section between the proximal and distal ends, wherein the smooth section is rotatably positioned within the second aperture; a first propellor section positioned on the shaft between the proximal end and the smooth section; and a second propellor section positioned on the shaft between the distal end and the smooth section, wherein rotation of the first and second propellor sections around a longitudinal axis of the shaft drives the surgical implant through bony tissue.

In other embodiments of the above surgical implant, a head of the anchor that is prevented by the first aperture from moving past the base section ring towards a distal end of the chassis.

In other embodiments of the above surgical implant, the smooth section of the shaft has a lesser diameter than the first and second propellor sections of the shaft.

In other embodiments of the above surgical implant, the first and second arms include sharpened distal ends to aid in cutting through bony tissue.

In other embodiments of the above surgical implant, the first and second arms of the chassis form a substantially U-shape extending from the base section.

In other embodiments of the above surgical implant, the first and second arms of the chassis each have a substantially U-shape profile with a concave portion of the U-shape facing inwards towards the shaft.

In other embodiments of the above surgical implant, the first and second arms, the base, and the bridge sections, form a retaining cavity for bony tissue.

In other embodiments of the above surgical implant, each of the first and second arms include at least one first aperture positioned and shaped to allow bony tissue to enter the retaining cavity.

In other embodiments of the above surgical implant, the first apertures are positioned near the bridge section.

In other embodiments of the above surgical implant, each of the first and second arms include at least one second aperture positioned and shaped to allow bony tissue to leave the retaining cavity if the pressure within the retaining cavity becomes too high.

In other embodiments of the above surgical implant, the second apertures are positioned near the base section.

In other embodiments of the above surgical implant, the bridge section is coupled to the first and second arms using a plurality of supporting arms, wherein spaces between the supporting arms enable bony tissue to enter the retaining cavity.

In other embodiments of the above surgical implant, the base section has a distal end and a proximal end, and the proximal end is wider than the distal end.

In other embodiments of the above surgical implant, the base section has a pyramidal shape.

In other embodiments of the above surgical implant, the base section has a cone shape.

In other embodiments of the above surgical implant, the base section includes protrusions that are positioned and angled to prevent the surgical implant from backing out of a surgical area.

In other embodiments of the above surgical implant, a plurality of distal-facing surfaces are tapered into cutting surfaces designed to harvest bony tissue as the shaft is rotated.

In other embodiments of the above surgical implant, a plurality of proximal-facing surfaces are angled to prevent the surgical implant from backing out of a surgical area.

In other embodiments of the above surgical implant, each of the first and second propellor sections include helical threads positioned about the shaft to form a plurality of helical root valleys between a plurality of crests, and the helical threads have a plurality of distal surfaces and a plurality of proximal surfaces.

In other embodiments of the above surgical implant, a plurality of generally radial pawls project from the distal surfaces to create a turbine-like thread-form shape, wherein the first plurality of pawls are angled with respect to the plurality of distal surfaces to allow for forward rotation of the plurality of threads and to resist backward rotation of the plurality of threads.

In other embodiments of the above surgical implant, at least one of the first and second propellor sections includes a first portion of the respective helical threads that are positioned distally from a second portion of the respective helical threads, wherein the first portion of helical threads has a larger major diameter than the second portion of helical threads.

In other embodiments of the above surgical implant, a circumferential rim projects from the plurality of proximal surfaces of the helical threads and is curved in a concave manner.

In other embodiments of the above surgical implant, the chassis is fenestrated to encourage bone growth after placement.

In other embodiments of the above surgical implant, the anchor includes an internal labyrinth having a main opening at the proximal end of the shaft and a plurality of openings along the shaft to allow an injection of bone growth material.

In other embodiments of the above surgical implant, the internal labyrinth has a tree-like structure wherein a plurality of tubules branch from a center cannula.

In other embodiments of the above surgical implant, the distal portion of the anchor includes a forward thread-form shape to assist in drilling through bony tissue during implant positioning.

In other embodiments, a surgical implant comprises a chassis that is configured to retain first and second anchors, the chassis having a central section with first, second, third, and fourth arms extending therefrom and including first, second, third, and fourth apertures, respectively, wherein the first and second arms are positioned on opposite sides of a proximal end of the chassis, the third and fourth arms are positioned on opposite sides of a distal end of the chassis, and the first and third arms are positioned on a first side of the chassis and configured to retain the first anchor, and the second and fourth arms are positioned on a second side of the chassis and configured to retain the second anchor; the first anchor having a first shaft having a proximal end and a distal end with a substantially smooth section between the proximal and distal ends, wherein the smooth section is rotatably positioned within the third aperture; a first propellor section positioned on the shaft between the proximal end and the smooth section; and a second propellor section positioned on the first shaft between the distal end and the smooth section, wherein rotation of the first and second propellor sections around a longitudinal axis of the first shaft in a first direction drives the surgical implant through bony tissue; and the second anchor having a second shaft having a proximal end and a distal end with a substantially smooth section between the proximal and distal ends, wherein the smooth section is rotatably positioned within the fourth aperture; a third propellor section positioned on the second shaft between the proximal end and the smooth section; and a fourth propellor section positioned on the second shaft between the distal end and the smooth section, wherein rotation of the third and fourth propellor sections around a longitudinal axis of the second shaft in a second direction drives the surgical implant through bony tissue.

In other embodiments of the above surgical implant, a head of each of the first and second anchors is prevented by the first and second apertures, respectively, from moving distally past the first and second arms.

In other embodiments of the above surgical implant, the smooth section of the first shaft has a lesser diameter than the first and second propellor sections of the first shaft, and the smooth section of the second shaft has a lesser diameter than the third and fourth propellor sections of the shaft.

In other embodiments of the above surgical implant, an upper surface and a lower surface of the chassis are substantially solid, and openings in the chassis face the first and second anchors to form a retaining cavity for bony tissue.

In other embodiments of the above surgical implant, a distal end of the chassis includes is sharpened to aid in cutting through bony tissue.

In other embodiments of the above surgical implant, the distal end of the chassis includes a plurality of supporting arms coupling the upper and lower surfaces, wherein spaces between the supporting arms enable bony tissue to enter the retaining cavity.

In other embodiments of the above surgical implant, each of the upper and lower surfaces of the chassis include at least one first aperture positioned and shaped to allow bony tissue to enter the retaining cavity.

In other embodiments of the above surgical implant, the first apertures are positioned near the distal end of the chassis.

In other embodiments of the above surgical implant, each of the upper and lower surfaces of the chassis include at least one second aperture positioned and shaped to allow bony tissue to leave the retaining cavity if the pressure within the retaining cavity becomes too high.

In other embodiments of the above surgical implant, the second apertures are positioned near the proximal end of the chassis.

In other embodiments of the above surgical implant, the base section has a distal end and a proximal end, and the proximal end is wider than the distal end.

In other embodiments of the above surgical implant, the first and second arms are larger than the third and fourth arms.

In other embodiments of the above surgical implant, the first and second arms have a pyramidal shape around their respective apertures.

In other embodiments of the above surgical implant, the first and second arms have a cone shape around their respective apertures.

In other embodiments of the above surgical implant, the first and second arms include protrusions that are positioned and angled to prevent the surgical implant from backing out of a surgical area.

In other embodiments of the above surgical implant, a plurality of distal-facing surfaces are tapered into cutting surfaces designed to harvest bony tissue as the first and second shafts are rotated.

In other embodiments of the above surgical implant, a plurality of proximal-facing surfaces are angled to prevent the surgical implant from backing out of a surgical area.

In other embodiments of the above surgical implant, each of the first, second, third and fourth propellor sections include helical threads positioned about the shaft to form a plurality of helical root valleys between a plurality of crests, and the helical threads have a plurality of distal surfaces and a plurality of proximal surfaces.

In other embodiments of the above surgical implant, a plurality of generally radial pawls project from the distal surfaces to create a turbine-like thread-form shape, wherein the first plurality of pawls are angled with respect to the plurality of distal surfaces to allow for forward rotation of the plurality of threads and to resist backward rotation of the plurality of threads.

In other embodiments of the above surgical implant, at least one of the first and second propellor sections includes a first portion of the respective helical threads that are positioned distally from a second portion of the respective helical threads, wherein the first portion of helical threads has a larger major diameter than the second portion of helical threads.

In other embodiments of the above surgical implant, at least one of the third and fourth propellor sections includes a first portion of the respective helical threads that are positioned distally from a second portion of the respective helical threads, wherein the first portion of helical threads has a larger major diameter than the second portion of helical threads.

In other embodiments of the above surgical implant, a circumferential rim projects from the plurality of proximal surfaces of the helical threads and is curved in a concave manner.

In other embodiments of the above surgical implant, the chassis is fenestrated to encourage bone growth after placement.

In other embodiments of the above surgical implant, each of the first and second anchors includes an internal labyrinth having a main opening at the proximal end of the shaft and a plurality of openings along the shaft to allow an injection of bone growth material.

In other embodiments of the above surgical implant, the internal labyrinth has a tree-like structure wherein a plurality of tubules branch from a center cannula.

In other embodiments of the above surgical implant, the distal portion of each of the first and second anchors includes a forward thread-form shape to assist in drilling through bony tissue during implant positioning.

1. A surgical implant comprising:
a chassis for aligning and holding a first propulsor and a second propulsor, wherein:
the first propulsor comprising:
a first longitudinal shaft having a first rotational axis; and
a clockwise oriented propeller positioned about a portion of the first longitudinal shaft, and
the second propulsor comprising:
a second longitudinal shaft having a second rotational axis; and
a counterclockwise oriented propeller positioned about a portion of the second longitudinal shaft,
wherein the clockwise oriented propeller and the counterclockwise oriented propeller each have:
helical threads positioned about a longitudinal portion of the respective shaft forming a plurality of helical root valleys between a plurality of crests of the helical threads, and the helical threads having a plurality of distal surfaces and a plurality of proximal surfaces;
a plurality of generally radial pawls projecting from the distal surfaces to create a turbine-like thread-form shape, wherein the first plurality of pawls are angled with respect to the plurality of distal surfaces to allow for forward rotation of the plurality of threads and to resist backward rotation of the plurality of threads;
a first section of the helical threads and a second section of the helical section of threads wherein the first section of helical threads is positioned distally from the second section of helical threads and the first section of helical threads has a larger major diameter than the second section of helical threads;
a circumferential rim projecting from the plurality of proximal surfaces of the helical threads and wherein the proximal surfaces of the helical threads are curved in a concave manner, and
an irregular porous lattice structure formed within the plurality of helical root valleys.

2. The surgical implant of claim 1, wherein the first propulsor has a first distal end shaped to cut through bony tissue during positioning and the second propulsor has a second distal end shaped to cut through bony tissue during positioning.

3. The surgical implant of claim 1, wherein the chassis is fenestrated to encourage bone growth after placement.

4. The surgical implant of claim 1, wherein the first propulsor and the second propulsor each define an internal labyrinth having a main opening at a proximal end and a plurality of openings along the propulsor to allow an injection of bone growth material.

5. The surgical implant of claim 4, wherein the internal labyrinth has a tree-like structure wherein a plurality of tubules branch from a center cannula.

6. The surgical implant of claim 1, wherein the first propulsor comprises a distal portion and a proximal portion, wherein the distal portion includes a forward distal thread-form shape to assist in drilling through bony tissue during implant positioning.

7. The surgical implant of claim 1, wherein the second propulsor comprises a distal portion and a proximal portion, wherein the distal portion includes a forward distal thread-form shape to assist in drilling through bony tissue during implant positioning.

8. A surgical implant comprising:
a chassis comprising
a first arm and a second arm projecting from a proximal base having a proximal aperture;
a distally positioned bridge coupling the first arm to the second arm having a distally positioned aperture; and
a propulsor having a first portion positioned within the distally positioned aperture and a second portion positioned within the proximal aperture;
wherein the propulsor has a propeller section comprising:
helical threads positioned about a longitudinal portion of a shaft forming a plurality of helical root valleys between a plurality of crests of the helical threads, and the helical threads having a plurality of distal surfaces and a plurality of proximal surfaces;
a plurality of generally radial pawls projecting from the distal surfaces to create a turbine-like thread-form shape, wherein the plurality of pawls are angled with respect to the plurality of distal surfaces to allow for forward rotation of the plurality of threads and to resist backward rotation of the plurality of threads;
a first section of the helical threads and a second section of the helical section of threads wherein the first section of helical threads is positioned distally from the second section of helical threads and the first section of helical threads has a larger major diameter than the second section of helical threads;
a circumferential rim projecting from the plurality of proximal surfaces of the helical threads and wherein the proximal surfaces of the helical threads are curved in a concave manner, and
an irregular porous lattice structure formed within the plurality of helical root valleys.

9. The surgical implant of claim 8, wherein the propulsor has a distal end shaped to cut through bony tissue during positioning and placement.

10. The surgical implant of claim 8, wherein the chassis is fenestrated to encourage bone growth after placement.

11. The surgical implant of claim 8, wherein the propulsor defines an internal labyrinth having a central opening at a proximal end and a plurality of openings along the propulsor to allow an injection of bone growth material.

12. The surgical implant of claim 11, wherein the internal labyrinth has a tree-like structure wherein a plurality of tubules branch from a center cannula.

13-33. (canceled)
